(19) United States
(12) Patent Application Publication
(10) Pub. No.: US 2012/0150070 A1
(43) Pub. Date: Jun. 14, 2012

(54) OVERTUBES FOR EUS FNA DRAINAGE

(76) Inventors: Shawn Ryan, Upton, MA (US); Joseph A. LEVENDUSKY, Groton, MA (US); Andrew HOLLETT, Waltham, MA (US)

(21) Appl. No.: 13/308,161

(22) Filed: Nov. 30, 2011

Related U.S. Application Data
(60) Provisional application No. 61/421,458, filed on Dec. 9, 2010.

(51) Int. Cl. A61B 17/34; A61B 10/02; A61B 17/00

(52) U.S. Cl. 600/567; 606/108; 604/164.04

(57) ABSTRACT

A device for performing multiple medical procedures comprises a needle extending from a proximal end which remains external to a body during use to a sharpened distal end configured for insertion to a target site within a body via a body lumen. A sheath is slidably receivable over the needle, the sheath extending from a proximal end which remains external to the body during use to a distal end. A stent is slidably received over the sheath, the stent including an anchoring mechanism configured for anchoring in a desired position within the body lumen.

### PRIORITY CLAIM

The application claims the priority to the U.S. Provisional Application Ser. No. 61/421,458, entitled “Overtubes for EUS FNA Drainage” filed Dec. 9, 2010. The specification of the above-identified application is incorporated herewith by reference.

### BACKGROUND

Endosonographers commonly use Endoscopic Ultrasound Fine Needle Aspiration (“EUS FNA”) devices for the diagnosis and staging of disease. EUS FNA is a highly effective diagnostic procedure, providing an ultrasound image which allows a physician to view a position of a distal portion of a needle in relation to a target tissue site. EUS FNA devices have been used, for example, in performing diagnostic procedures such as needle biopsies in the gastrointestinal tract or other lumens of a living body (e.g., accessed via a naturally occurring body orifice).

### SUMMARY OF THE INVENTION

The present invention is directed to a device and method for performing multiple medical procedures. The device according to the invention a needle extending from a proximal end which remains external to a body during use to a sharpened distal end configured for insertion to a target site within a body via a body lumen. A sheath is slidably receivable over the needle, the sheath extending from a proximal end which remains external to the body during use to a distal end. A stent is slidably received over the sheath, the stent including an anchoring mechanism configured for anchoring in a desired position within the body lumen.

### BRIEF DESCRIPTION OF THE DRAWINGS

FIG. 1 shows an overtube according to a first exemplary embodiment of the present invention;

FIG. 2 shows an overtube according to a second exemplary embodiment of the present invention;

FIG. 3 shows an overtube according to a third exemplary embodiment of the present invention;

FIG. 4 shows an overtube according to a fourth exemplary embodiment of the present invention;

FIG. 5 shows an overtube according to a fifth exemplary embodiment of the present invention;

FIG. 6 shows an overtube according to a sixth exemplary embodiment of the present invention;

FIG. 7 shows an overtube according to a seventh exemplary embodiment of the present invention;

FIG. 8 shows an overtube according to a seventh exemplary embodiment of the present invention in a first operative configuration; and

FIG. 9 shows the overtube of FIG. 8 in a second operative configuration.

### DETAILED DESCRIPTION

The present application may be further understood with reference to the following description and the appended drawings, wherein like elements are referred to with the same reference numerals. The present invention relates to devices for adapting EUS FNA devices to perform therapeutic procedures in conjunction with their diagnostic capabilities. For example, an EUS FNA device according to an embodiment of the present invention may be employed to perform a therapeutic drainage procedure without requiring the removal of the EUS FNA device or the insertion of a separate device. Exemplary embodiments of the present invention are directed to a sheath provided over an EUS FNA device at least partially encased in an overtube exhibiting predetermined characteristics configured and adapted to aid in retention of the EUS FNA device at a target position within the body. Specifically, a device according to the present invention employs an FNA needle to guide the insertion and placement of a sheath and anchoring means to a target tissue site within the body. The sheath and/or anchoring means, which may be formed as an overtube extending over the sheath, are positioned coaxially over the needle and advanced over the needle into the body once the needle has been advanced to a target tissue site. Alternately, the positioning and deployment may be performed prior to or after use of the EUS FNA needle. In one example, the overtube is formed as a stent having one or more projections extending therefrom to engage surrounding tissue at the target tissue site to anchor the device in place. As described below, the stent is configured and dimensioned so that the projections are prevented from deploying until the stent has reached the target position. The exemplary embodiments according to the present invention permit the insertion of multiple tools into a living body with an EUS FNA device, thus reducing the total time necessary to complete a medical procedure while, at the same time, reducing the difficulty of the procedure for a physician. As used in this application, the term proximal refers to a direction approaching the physician or other user and the term distal refers to a direction approaching a target tissue site in the body.

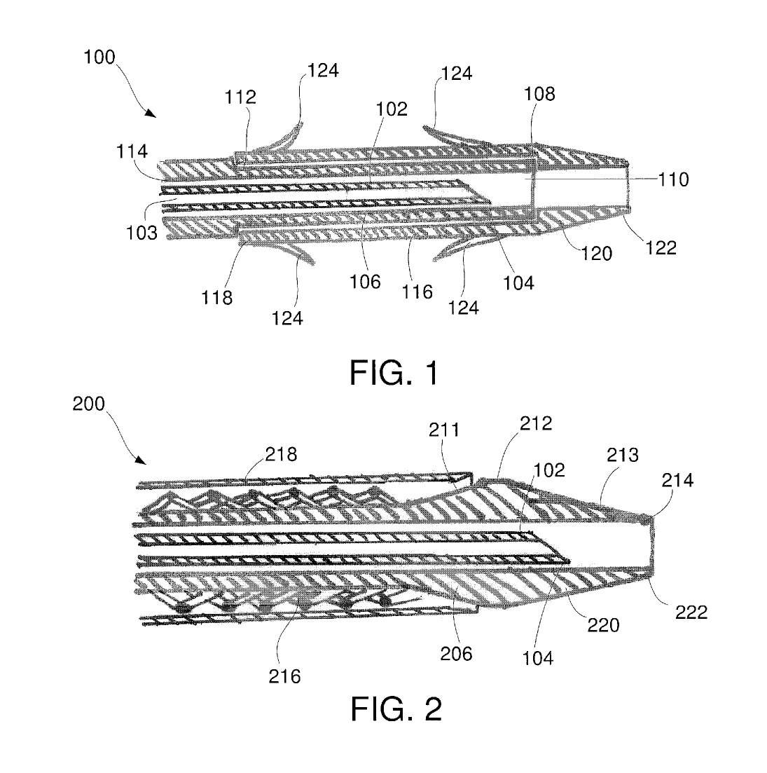

As shown in FIG. 1, a device 100 according to a first exemplary embodiment of the present invention comprises a substantially cylindrical EUS FNA needle extending from a proximal end (not shown) to a sharpened distal end 104 which is inserted into the body to a target portion of tissue. As would be understood by those skilled in the art, the proximal end which may be coupled to a handle or actuating member (not shown) remains outside the body during use. A lumen 103 extending through the needle 102 is open at the proximal and distal ends and a flexible yet resilient sheath 106 is slidably received over the needle 102. Thus an inner diameter of the sheath 106 is larger than an outer diameter of the needle 102. In an exemplary embodiment, the sheath 106 may be formed of polyethylene (“PE”), polytetrafluoroethylene (“PTFE”) or another material exhibiting desired properties. A length of the sheath 106 is selected so that, when slid over the needle 102 in an operative configuration, the sheath 106 extends from a proximal end external to the body to a distal end 108 projecting distally beyond the distal end 104 of the needle 102. The distal end 108 of the sheath 106 has an opening 110 permitting slidable movement of the needle 102 therethrough. A distal portion of the sheath 106 formed as a reduced diameter portion 112 has an outer diameter smaller than that of the proximal portion of the sheath 106. In another embodiment, the distal portion of the sheath 106 may be tapered. An inner diameter 114 of the sheath 106, however, is preferably kept substantially constant to prevent trauma to sampled tissue or other biological matter and to prevent the needle from being caught within the sheath 106. A stent 116 is received over the reduced diameter portion 112. The reduced outer diameter of the reduced diameter portion 112 allows an outer diameter of a proximal portion 118 of the stent 116 to be substantially similar to an outer diameter of the reduced diameter portion 112, the reduced diameter portion 112 being configured to slidably receive the stent 116 thereover. In another embodiment of the invention (not shown), the stent 116 may be held in position by a mechanism known in the art including, but not limited to a friction-fit, surface roughening within the sheath 106, a light adhesive or a tacky coating (e.g., silicone, etc.). As would be understood by those skilled in the art, the stent 116 may be formed of any of a variety of suitable materials including, but not limited to, extruded plastic, injection molded plastic, laser cut metal, braided metal wire, woven metal wire and coated metal wire. A distal portion 120 of the stent 116 has a taper reducing in diameter to a distal end 122 so that a diameter of the stent 116 is smallest at the distal end 122. An inner diameter of the distal portion 120 is also smaller than an inner diameter of the proximal portion 118 of the stent 116. In one embodiment, the inner diameter of the distal portion 120 of the stent 116 is substantially the same as the inner diameter of the sheath 106 to provide a uniform path for the movement of the needle 102 therethrough.

The stent 116 further comprises a plurality of projections 124 extending from the proximal portion 118. The projections 124 are formed as cut-out flaps from an outer wall of the stent 116 and are oriented to extend out from the proximal portion 118 such that the device 100 may be advanced through a channel of an endoscope without causing any interference. The projections 124 are substantially soft and flexible to prevent any tissue damage. In another embodiment, the projections 124 may be configured and dimensioned so that, in a deployed position, they engage tissue surrounding the stent 116. Specifically, the projections 124 may be movable from an insertion position (not shown) in which the projections 124 lie substantially flush against an outer wall of the stent 116 to the deployed position shown in FIG. 1 upon actuation by an actuating mechanism (not shown). In one embodiment, the stent 116 may be further encased in a capture tube (not shown) preventing contact between the projections 124 and non-targeted tissue (e.g., tissue along the path over which the device is inserted to the target position) until it is desired to move the stent 116 to the deployed position. Specifically, the projections 124 may be elastic and biased toward the deployed position so that a compressive force applied by the capture tube retains the projections flush against the stent 116 during insertion to the target tissue site. After the stent 116 has been positioned as desired relative to the target tissue site, the capture tube 106 is retracted proximally exposing the stent 116 and permitting the projections 124 to move under their natural bias to the deployed position in which they project outward from the stent 116. Contact of the projections 124 with near-lying tissue anchors the stent 116 and device 100 within the body. It is noted that although the projections 124 are shown in FIG. 1 with particular shapes and orientations (i.e., two pairs of projections pointed toward one another), any other shapes and/or orientations may be employed without deviating from the spirit and scope of the present invention. Furthermore, the projections 124 may be provided in any number and in any pattern without deviating from the spirit and scope of the present invention. Additionally, the projections 124 may optionally be moved to the insertion position by a wrapped filament, adhesive, friction-fit, etc.

In accordance with an exemplary method according to the present invention, the needle 102 is first inserted into a body lumen (e.g., via a bodily orifice) and advanced through the lumen to a target position. The needle 102 which may include a stylet (not shown) inserted therethrough, as would be understood by those skilled in the art may be positioned with the aid of a guidewire previously inserted into the body, through an endoscope inserted into the body, etc., as those skilled in the art will understand. The needle 102 may be guided through a lumen of an endoscopic viewing instrument or by any other technique known in the art. Once the needle 102 has reached a target location, the needle 102 may be actuated as desired to move the sharpened tip 104 into target tissue to capture biological matter therein in connection with an exemplary sampling procedure, as those skilled in the art will understand. The needle 102 may then be maintained in this target location or moved to a new target location at which it is desired to deploy the stent 116. The sheath 106 with the stent 116 positioned thereover may then be slid over the needle 102 until the distal end 122 of the stent 116 is positioned distally of the sharpened tip 104. The stent 116 is then anchored in the desired position by engaging the projections 124 in adjacent tissue, as described in greater detail earlier. Retraction of the stent 116, sheath 106 and needle 102 from the body may be done simultaneously or if so desired, individually.

As shown in FIG. 2, a device 200 according to a first alternate embodiment of the present invention is formed substantially similarly to the device 100 of FIG. 1 with the exception of a stent 216 provided therewith. Specifically, the needle 102 is encased within a sheath 206 comprising a proximal portion 208 of with inner and outer diameters that are substantially uniform along the length of a proximal portion thereof. A distal portion 210 of the sheath 206 may include a first tapered portion 211 with an outer diameter increasing in a distal direction to an intermediary portion 212 having a one of a uniform outer diameter and a varying outer diameter (not shown). A distal end of the intermediary portion 212 may be connected to a second tapered portion 213 having an outer diameter decreasing to a distal end 214. An inner diameter of the sheath 206 remains constant throughout its length, regardless of the changes in the outer diameter in the distal portion 210. It is further noted that although the sheath 206 is depicted having tapered portions, this embodiment is exemplary only and any manner of the tip may be provided without deviating from the scope of the invention.

A metal or polymer stent 216 received over the proximal portion 208 of the sheath 206 may be formed as a woven filament biased to a radially expanded configuration. The stent 216 may be formed with any configuration including, but not limited to, laser cut, braided, knitted, wound, twisted, knotted, coiled, solid tube stents, stent grafts and the like. The stent 216 is encased in a capture tube 218 sized and shaped to prevent the stent 216 from contacting non-targeted adjacent tissue during insertion through the body. An outer diameter of the capture tube 218 is substantially similar to or smaller than an outer diameter of the intermediary portion 212, thus preventing the capture tube 218 from being moved distally beyond the sheath 206. As discussed in greater detail above, once the needle 106 has been advanced to a target portion in the body, the sheath 206 and capture tube 218 are advanced distally thereover to a target position. The capture tube 218 is then moved proximally relative to the stent 216, permitting radial expansion thereof to anchor the stent 216 at the desired location in the body. That is, the stent 216 is biased to radially expand beyond the diameter at which it is constrained within the tube 218 so that removal of the compressive force applied thereby permits the metal stent 216 to expand in the body to engage surrounding tissue. As those skilled in the art will understand, an outer diameter of the expanded stent 216 is preferably selected to conform to the dimensions of the body lumen to be treated so that the stent 216 will be anchored in the desired position without undue trauma to the surrounding tissue.

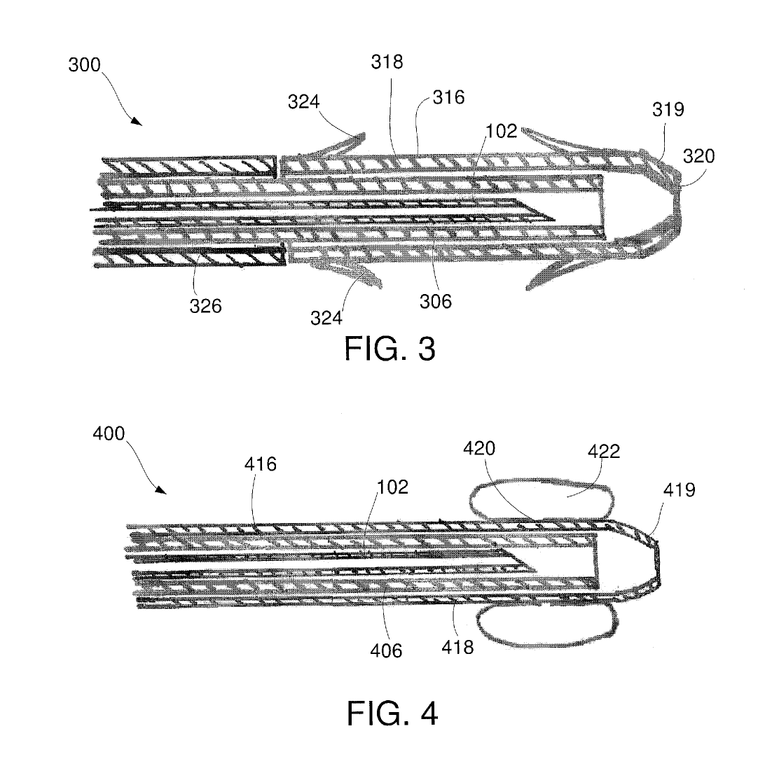

As shown in FIG. 3, a device 300 according to a second alternate embodiment of the present invention is formed substantially similarly to the device 100 of FIG. 1 with the exception of a stent 316 and the addition of a push tube 326, as will be described in greater detail hereinafter. The needle 102 is encased within a sheath 306 having a uniform inner and outer diameter. A stent 316 received over a distal portion of the sheath 306 comprises a proximal portion 318 having a uniform outer diameter and a distal portion 319 tapering down in diameter to a distal end 320. The stent 316 comprises projections 324 formed substantially similarly to the projections 124 of the stent 116. The distal portion 319 of the stent 316 is substantially flexible so that it may be radially expanded to allow the distal portion 319 to be slid over the proximal end of the sheath 306. That is, once the needle 102 and the sheath 306 have been advanced to a target position in the body, the distal portion 319 of the stent 316 is expanded to fit over the proximal end of the sheath 306. The stent 316 is then pushed distally over the sheath 316 using the push tube 326 until the stent 316 has reached the configuration shown in FIG. 3. In another embodiment, the distal end 320 of the stent 316 may be formed with a diameter permitting the sheath 306 to slide therethrough without necessitating expansion of the distal portion 319. The push tube 326 preferably has inner and outer diameters substantially the same as the stent 316 and is long enough so that, the proximal end of the push tube 326 remains outside the body when the stent 316 reaches the target position at the distal end of the sheath 306. Once a physician has confirmed that the stent 316 has reached a target site in the body (e.g., under endoscopic guidance, etc.), the push tube 326, sheath 306 and needle 102 may be retracted proximally and removed from the body. The stent 316 is held in place within the body by anchoring of the projections 324 with the tissue. In another embodiment, the needle 102 may first be retracted from the body and the device 300 and followed by a retraction of the push tube 326 and sheath 306.

As shown in FIG. 4, a device 400 according to a third alternate embodiment of the present invention is substantially similar to the device 300 of FIG. 3 except that the stent 316 and push tube 326 are replaced in this device by a unitary overtube 416, as will be described in greater detail hereinafter. A sheath 406 slidably receivable over the needle 102 has inner and outer diameters substantially uniform along its length and the overtube 416 which is slidably disposed over the sheath 406 and also inner and outer diameters substantially uniform over a portion of its length extending from a proximal end thereof to a proximal end of a distal portion 419. The distal portion 419 is tapered similarly to the distal portion 319 of FIG. 3. In one embodiment, the overtube 416 may be configured to be inserted into the body simultaneously with the sheath 406. A balloon 422 is mounted to the overtube 416 adjacent to the distal portion 419 and an inflation lumen 418 extends within a wall of the overtube 416 from a proximal port (not shown) to a distal port opening into the balloon 422 so that a fluid or gas supplied to the proximal port may inflate the balloon 422. Once the device 400 has been positioned at a target location in the body, the balloon 422 is inflated via the inflation lumen 418 to anchor the device 400 in place. The balloon 422 may be deflated after completion of a target procedure and subsequently withdrawn from the body. In another embodiment, the balloon 422 may be used as an anchor for a stent, including any of the stents disclosed in the present application. In another embodiment, the balloon 422 may used as a balloon catheter to aid in dilating a portion of a body lumen.

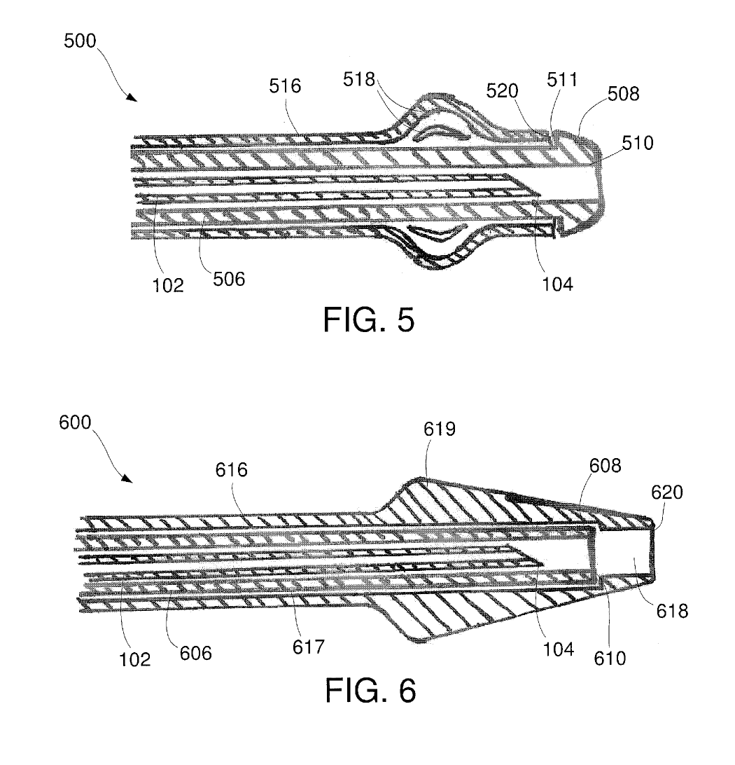

As shown in FIG. 5, a device 500 according to a fourth embodiment of the invention is formed substantially similarly to the device 100 of FIG. 1 with the exception of a dilation overtube 516 provided thereover. Specifically, the device 500 comprises a sheath 506 slidably receivable over the needle 102. The sheath 506 which is substantially similar to the sheath 106 of FIG. 1 has a distal end 508 including an increased diameter portion 510 an outer diameter of which is greater than that of proximal portions of the sheath 506. A lip 511 defined by the increased diameter portion 510 extends at an angle substantially perpendicular to a longitudinal axis of the sheath 506 and serves as an interface with the dilation overtube 516. The distal end 508 of the sheath 506 includes an opening sized and shaped to permit the sharpened tip 104 of the needle 102 to project distally therefrom in an operative configuration. Similarly to the overtube 416 of FIG. 4, the dilation overtube 516 is formed of a substantially flexible yet resilient material and comprises a plurality of circumferentially aligned slots 518 formed on a distal portion thereof. In an exemplary embodiment, the slots 518 are positioned so that, when the overtube 516 is slid over the sheath 506 until a distal end 520 thereof contacts the lip 511, the slots 518 are proximal of a target portion of tissue to anchor the device 500 in place. That is, the slots 518 allow for radial expansion of the overtube 516 when actuated by an actuating mechanism (not shown) located at a proximal end of the device 500. In one embodiment of the present invention, the actuation mechanism may simply be a means to apply a distally directed force to the overtube 516 to advance the overtube 516 distally over the sheath 506 once the sheath 506 has been advanced to a target position over the needle 102. When a distal end of the overtube 516 contacts the lip 511, further application of a distally directed force causes the overtube 516 to fold over at the slots 518, buckling and radially expanding as shown in FIG. 5. A position of a proximal portion of the overtube 516 which remains external to the body may then be locked using any known mechanism to prevent the radially expanded distal portion thereof from becoming straightened. Those skilled in the art will understand that such a mechanism is not required and may alternately be omitted by forming the overtube 516 of a material that maintains the radially expanded configuration until application of a proximally directed force causes the slots 518 to once again retract to lie flush against the outer periphery of the sheath 506. For example, the overtube 516 may be formed of polypropylene, polyethylene, PTFE, FEP, polyurethane, any shape memory polymer, Nitinol or any other shape memory alloys, as those skilled in the art will understand. In another embodiment of the present invention, the actuating mechanism may include a pullwire (not shown) extending from an actuatable handle at a proximal end of the device 500 to a distal end connected to a portion of the overtube 516 adjacent to the slots 518, so that proximal retraction of the pullwire builds tension to the slots 518 and subsequently causes a radial expansion of the overtube 516 at the slots 518. In yet another embodiment, the sheath 506 may be retracted proximally to apply a proximally directed force to buckle the overtube 516.

As shown in FIG. 6, a device 600 according to a fifth embodiment of the invention is formed substantially similarly to the device 500 of FIG. 5 except for a dilation overtube 616 provided thereover. Specifically, the needle 102 of the device 600 has a substantially coaxial sheath 606 provided thereover extending from a proximal end (not shown) which remains outside the body when the distal end 608 thereof is inserted into the body to a desired location adjacent to target tissue to be treated. The distal end 608 has an opening 610 to permit slidable movement of the needle 102 into and distally therefrom. The overtube 616 is slidably receivable over the sheath 606 and has a substantially cylindrical body with a lumen 617 extending therethrough. The lumen 617 is also substantially cylindrical and is sized to permit the overtube 616 to slide over the sheath 606 with a minimal amount of friction. A distal end of the lumen 617 comprises a reduced diameter portion 618 having an inner diameter smaller than an outer diameter of the sheath 606. Thus, the reduced diameter portion 618 prevents the sheath 606 from sliding distally out of the overtube 616 while still permitting movement of the needle 102 distally out of the overtube 616. An outer periphery of a distal portion 619 of the overtube 616 is formed with a taper configured and dimensioned to dilate a path created by the needle 102 as the sheath 606 and overtube 616 are slid distally over the needle 102 further into the body. The distal portion 619 of the overtube 616 is configured with an outer diameter increasing at a predetermined rate from the distal end 620 to a position separated proximally therefrom by a predetermined distance. In an exemplary embodiment, this distance may be selected so that a maximum diameter of the overtube 616 at a proximal end of the distal portion 619 is substantially similar to an outer diameter of a body lumen in which the device 600 is inserted to prevent trauma to the body lumen. The proximal end of the distal portion 619 may be formed with a smooth transition from the uniform outer diameter portion of the overtube 616 to the distal portion 619, the smooth transition minimizing trauma to tissue when inserted into the body. In an operative configuration, the sheath 606 and overtube 616 are simultaneously inserted into the body over the needle 102.

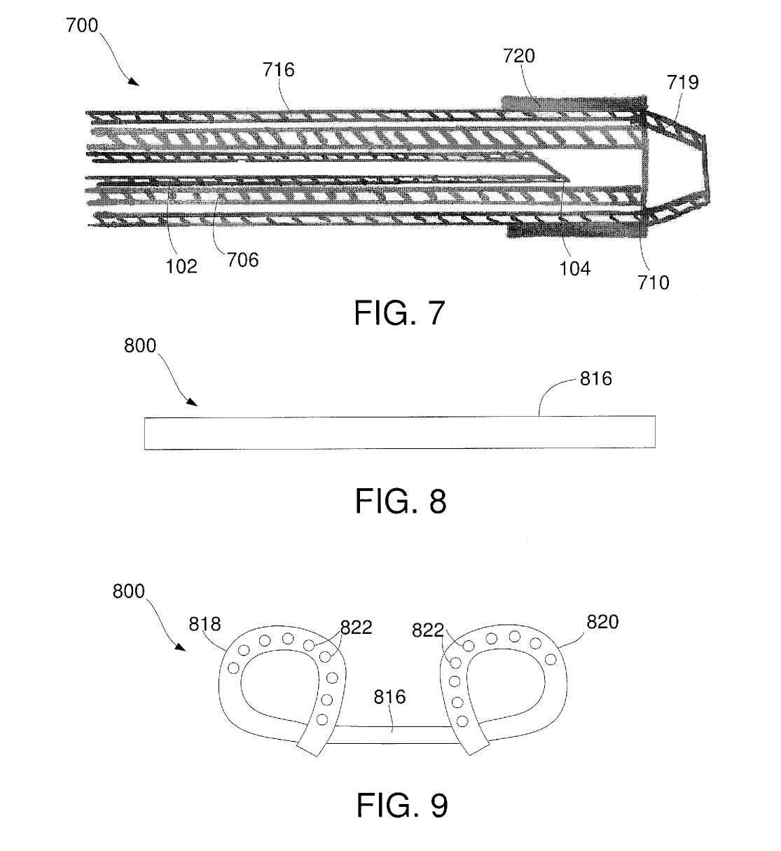

As shown in FIG. 7, a device 700 according to a sixth alternate embodiment of the present invention is formed substantially similarly as the device 400 of FIG. 4 with the exception of an overtube 716. Specifically, the device 700 comprises the needle 102 and a sheath 706 provided thereover, the sheath 706 being substantially cylindrical in shape and extending to a distal end 710 having an opening. The overtube 716 is formed substantially similarly as the overtube 416 of FIG. 4 and has a substantially cylindrical shape with a tapered distal portion 719 similar to the tapered distal portion 419. An outer periphery of a distal portion of the overtube 716 located just proximally of the tapered distal portion 719 includes an RF contact electrode 720. The RF contact electrode 720 extends circumferentially over the overtube 716. The RF contact electrode 720 is configured to cut and/or cauterize a path created by the needle 102 to aid in slidable movement of the overtube 716 into the body. In an operative configuration, the RF contact electrode 720 may be energized during insertion into the body to aid in distal movement of the overtube 716. The exemplary RF contact electrode 720 according to the present embodiment may also be employed in combination with any of the embodiments disclosed earlier without deviating from the spirit and scope of the present invention.

As shown in FIGS. 8 and 9, a device 800 according to a seventh alternate embodiment of the invention, comprises a stent 816 movable from a first elongated configuration shown in FIG. 8 to a second deployed pigtail configuration shown in FIG. 9. The exemplary stent 816 is configured to be slidably received over a sheath, such as the sheath 306 of FIG. 3. In an operative configuration, the stent 816 is advanced distally off of the sheath 306 and, due to its biased curvature, assumes the pigtail shape of FIG. 9, with one or both of the proximal and distal ends 818, 820 thereof assuming a curled configuration. As those skilled in the art will understand, this configuration aids in retaining the stent in position within a body lumen. The stent 816 also comprises one or more drainage ports 822 disposed on the proximal and distal ends 818, 820. The drainage ports 822 may alternately be provided over any portion of the stent 816 without deviating from the scope of the invention.

The exemplary devices according to the invention permit a physician or other user to perform numerous procedures within the body without having to remove an endoscope therefrom. Specifically, an EUS FNA needle may be used to sample tissue from a target location in the body (e.g. a GI tract). The needle may subsequently be withdrawn proximally within the endoscope to permit stent placement, balloon dilation, ablation or another procedure via the endoscope. The needle or another device may then be redeployed through the endoscope to perform another target procedure.

Various modifications may be made to the disclosed devices without deviating from the scope of the present invention. For example, a device according to another embodiment of the invention comprises an anchoring mechanism configured to be placed over the EUS FNA needle before deployment in the body. As those skilled in the art will understand, this device permits the use of different combinations of medical devices and anchoring mechanisms. Additionally, such a design permits the addition of alternate medical devices onto or within the device prior to insertion thereof into the body. Any of the stents disclosed in the present application may be preloaded over an endoscope and/or needle and subsequently deployed in the body after completion of a first target procedure by the needle or other medical device, as those skilled in the art will understand.

It will be apparent to those skilled in the art that various modifications can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A device for performing multiple medical procedures, comprising:
a needle extending from a proximal end which remains external to a body during use to a sharpened distal end configured for insertion to a target site within a body via a body lumen;
a sheath slidably receivable over the needle, the sheath extending from a proximal end which remains external to the body during use to a distal end; and
a stent slidably received over the sheath, the stent including an anchoring mechanism configured for anchoring in a desired position within the body lumen.

2. The device of claim 1, wherein the anchoring mechanism is formed as one of a projection jutting radially outward from an overtube of the stent, a braided metal wire, a woven metal wire, a coated metal wire and an inflatable balloon, the overtube being provided over at least a portion of the sheath.

3. The device of claim 2, wherein the projections are formed as cut-out flaps from an outer wall of the stent.

4. The device of claim 1, further comprising a substantially cylindrical push tube configured and dimensioned to advance the stent distally over the sheath, the push tube having a proximal end which remains external to the body during use and a distal face configured to apply a distally directed force to the stent.

5. The device of claim 1, further comprising a capture tube provided over the stent, the capture tube configured and dimensioned to apply a radially compressive force to the stent to encase the anchoring mechanism therewithin, proximal movement of the capture tube relative to the anchoring mechanism permitting radial expansion of the anchoring mechanism.

6. The device of claim 1, wherein the stent is configured and dimensioned to be coaxially slidable over the sheath.

7. The device of claim 2, wherein the overtube is coaxially slidable over the sheath.

8. The device of claim 7, wherein the overtube comprises an inflation lumen, the inflation lumen being open to the anchoring mechanism, wherein the anchoring mechanism is an inflatable balloon.

9. The device of claim 7, wherein the overtube comprises a slot extending through a distal portion thereof, the slot defining a weakened point of the overtube, which, when subjected to an axially compressive force of a predetermined magnitude, causes radial expansion of the overtube.

10. The device of claim 7, wherein the stent comprises a curled biased configuration and wherein proximal retraction of the overtube from the stent causes movement of the stent to the biased configuration.

11. The device of claim 7, further comprising a radio-frequency contact electrode provided on a distal portion of the overtube, the electrode aiding in insertion of the overtube into the body lumen by one of cutting and cauterizing adjacent portions of tissue during insertion.

12. The device of claim 1, wherein the stent comprises a drainage port provided on a distal portion of the stent.

13. The device of claim 1, wherein a distal portion of the stent is tapered to aid in insertion into the body.

14. A device for performing fine-needle aspiration, comprising:
a needle extending from a proximal end which remains external to a body during use to a sharpened distal end configured for insertion to a target site within a body via a body lumen;
a sheath slidably receivable over the needle, the sheath extending from a proximal end which remains external to the body during use to a distal end; and
an anchoring mechanism slidably received over the sheath, the anchoring mechanism including an anchoring element at a distal end thereof to anchor the needle in a desired position within the body lumen to act as a guide over which a therapeutic instrument may be advanced to treat tissue adjacent to the target site.

15. The device of claim 14, wherein the anchoring mechanism is a stent.

16. The device of claim 15, wherein the stent further comprises an overtube coaxially slidable over the sheath.

17. The device of claim 16, wherein anchoring mechanism is formed as one of a projection jutting radially outward from the overtube, a braided metal wire, a woven metal wire, a coated metal wire and an inflatable balloon.

18. A method for inserting a diagnostic instrument into a living body, comprising:
inserting a flexible needle through a body lumen into target portion of tissue within a living body to obtain a sample of target tissue;
inserting a sheath coaxially over the needle along a path created by the needle, the sheath having a proximal end which remains external to the body and a distal end having an opening to permit slidable movement of the needle distally therefrom; and
deploying a therapeutic device from a distal portion of the sheath to treat tissue adjacent to the target tissue.

19. The method of claim 18, wherein the medical device includes a stent configured and dimensioned to anchor a distal portion of the needle at a target position.

20. The method of claim 19, wherein the stent is received on a distal portion of the sheath.

21. The method of claim 19, further comprising the step of advancing a push tube distally over the sheath to move the stent to a desired position at a distal end of the sheath.

22. The method of claim 19, wherein the stent is encased within a protective overtube, the method further comprising the step of:
retracting the overtube proximally to expose the stent to surrounding tissue, retraction of the overtube permitting the stent to move under a natural biased to a radially expanded configuration.

23. The method of claim 22, wherein the needle and sheath are retracted simultaneously with the overtube.

24. The method of claim 18, further comprising the steps of:
advancing an overtube over the sheath, the overtube having an inflation lumen extending therethrough and open to an inflatable balloon at a distal end thereof; and
inflating the inflatable balloon after the overtube has been advanced to a target position.

25. The method of claim 18, further comprising the step of:
advancing an overtube over the sheath, the overtube having a plurality of circumferentially aligned slots extending therethrough; and
actuating the overtube to cause a radial expansion of the slots, the radial expansion anchoring the overtube at a target position in the body.

26. The method of claim 25, wherein the overtube is actuated by applying a distally directed force to the overtube moving a distal end of the overtube against a lip on a distal end of the sheath, redirecting a portion of the distally directed force to a radially expansive force.

27. The method of claim 25, wherein the overtube is actuated by applying a proximally directed force to the overtube via pullwire extending from a proximal handle which remains external to the body in an operative configuration to a distal end connected to a portion of the overtube adjacent the slots.

28. The method of claim 25, wherein the sheath is sized so that, when inserted into a target body lumen, the lumen is dilated.

29. The method of claim 18, further comprising the step of advancing an overtube over the sheath, the overtube having a radio-frequency contact patch provided on a distal portion thereof, the patch aiding in insertion of the overtube by one of cutting and cauterizing adjacent portions of tissue during insertion into the body.
